(19) United States
(12) Patent Application Publication
(10) Pub. No.: US 2025/0256078 A1
(43) Pub. Date: Aug. 14, 2025

(54) 3D DISPENSER FOR BIO-APPLICATIONS

(71) Applicant: Tides Medical, LLC., Lafayette, LA (US)

(72) Inventor: Mohammadali Babak Safavieh, North Andover, MA (US)

(73) Assignee: Tides Medical, LLC., Lafayette, LA (US)

(21) Appl. No.: 18/913,323

(22) Filed: Oct. 11, 2024

Related U.S. Application Data
(60) Provisional application No. 63/589,706, filed on Oct. 12, 2023.

(51) Int. Cl. A61M 35/00; B33Y 10/00; B33Y 30/00

(52) U.S. Cl. CPC A61M 35/003; B33Y 10/00; B33Y 30/00; A61M 2205/3606

(57) ABSTRACT

This disclosure generally relates to devices for printing a graft onto a wound. In some embodiments, the devices comprise one or more reservoirs adapted to receive and/or contain a biological material or a synthetic material. The devices may further comprise a cooling component adjacent the reservoir configured to cool the internal portion of the one or more reservoirs at greater than or equal to −30° C. and less than or equal to 10° C., according to other embodiments. In some embodiments, the device comprises a dispenser configured to dispense the biological and/or synthetic material onto a surface of the wound. In some embodiments, the device preserves the native structure of the biological and/or synthetic material after deposition. This disclosure also provides methods for printing a graft onto a wound e.g., using the devices contemplated herein.

### FIELD

The present disclosure generally relates to the field of bioprinting grafts for wound healing and other indications.

### BACKGROUND

Chronic wounds, e.g., non-healing wounds, are wounds that fail to proceed through the normal phases of wound healing in an orderly and timely manner. The primary causes of chronic wounds include bacterial infection, tissue ischemia, and/or an underlying medical condition such as diabetes or an impaired immune system. Further, chronic wounds are associated with significant morbidity (e.g., amputation) and mortality and represent a major medical and financial burden. Treatments that improve healing of chronicle wounds are needed.

### SUMMARY

The subject matter of the present disclosure involves, in some cases, interrelated products, alternative solutions to a particular problem, and/or a plurality of different uses of one or more systems and/or articles.

Aspects of the present disclosure generally relate to devices for the deposition of a graft. In some embodiments, the devices contemplated herein are for the deposition of a biological material and/or synthetic material onto a surface of a subject. In some embodiments, the devices comprise one or more reservoirs adapted to receive and/or contain one or more biological materials or one or more synthetic materials. In some embodiments, the devices comprise a cooling component adjacent the one or more reservoirs, wherein the cooling component is configured to keep an internal portion of the one or more reservoirs at greater than or equal to −30° C. and less than or equal to 10° C. In some embodiments, the devices further comprise a dispenser adjacent the one or more reservoirs, wherein the dispenser is configured to dispense the one or more biological materials or the one or more synthetic materials onto the surface of the subject.

Other aspects of the present disclosure generally relate to methods of printing a graft using one or more of the devices contemplated herein. In some embodiments, the methods comprise printing a graft onto a wound of a subject. In some embodiments, the methods comprise contacting a device to the wound and moving said device across said wound in a user-defined path. In some embodiments, the methods further comprise depositing one or more biological materials or one or more synthetic materials, from the device onto the surface of said wound as the device moves along the user-defined path. In some embodiments, the device is configured to maintain the one or more biological materials or the one or more synthetic materials at a temperature of greater than or equal to −30° C. and less than or equal to 10° C. prior to deposition.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, devices. In still another aspect, the present disclosure encompasses methods of using one or more of the embodiments described herein, for example, devices.

Other advantages and novel features of the present disclosure will become apparent from the following detailed description of various non-limiting embodiments of the disclosure when considered in conjunction with the accompanying figures.

### BRIEF DESCRIPTION OF DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A shows a schematic diagram of an exemplary device, according to one set of embodiments.

FIG. 1B shows a computer automated drawing (CAD) of an exemplary device comprising a first reservoir (e.g., cartridge 1), a second reservoir (e.g., cartridge 2), mixing component (e.g., microfluidic mixer cartridge), and a nozzle (e.g., nozzle dispenser), according to one set of embodiments.

FIG. 2 show a computer automated drawing (CAD) of an exemplary device comprising a circuit board, a start button, and an inlet to the cooling jacket according to one set of embodiments.

FIGS. 3A-3D show computer automated drawings (CAD) of exemplary microfluidic-based mixing components comprising a first convergence-divergence passive mixer and a second serpentine mixer, according to one set of embodiments.

FIG. 4 shows a computer automated drawing (CAD) of the interface between the mixing component and the cooling system (e.g., Peltier system) of an exemplary device, according to one set of embodiments.

FIG. 5 shows the exploded view of a handheld device comprising two reservoir system with all the components used according to one set of embodiments.

FIG. 6 shows the computer automated drawing (CAD) illustrating the use of the two-reservoir device to deposit mixed biological materials or synthetic materials onto a surface using a serpentine-based user defined path, according to one set of embodiments.

FIG. 7A is a computer automated drawing (CAD) of an exemplary device, according to some embodiments of the present disclosure. FIG. 7B shows a cross-sectional view of the exemplary device shown in 6A, according to one set of embodiments.

FIG. 8 shows a computer automated drawing (CAD) illustrating the use of the single reservoir device to deposit a biological material or synthetic material onto a surface using a serpentine-based user defined path, according to one set of embodiments.

FIG. 9A shows a photograph of an exemplary device of the present disclosure. FIG. 9B shows photograph illustrating the use of the device to deposit a biological material onto a substrate, according to one set of embodiments.

### DETAILED DESCRIPTION

This disclosure generally relates to devices for printing a graft (e.g., a wound dressing) onto a wound. In some embodiments, the devices comprise one or more reservoirs adapted to receive and/or contain a biological material or a synthetic material. The devices may further comprise a cooling component adjacent the reservoir configured to cool the internal portion of the one or more reservoirs at greater than or equal to −30° C. and less than or equal to 10° C., according to other embodiments. In some embodiments, the device comprises a dispenser configured to dispense the biological and/or synthetic material onto a surface of the wound. In some embodiments, the device preserves the native structure of the biological and/or synthetic material after deposition. This disclosure also provides methods for printing a graft onto a wound using the devices contemplated herein.

Without wishing to be bound by any particular theory, it is generally understood that wounds are more susceptible to healing in a moist, clean, and warm environment. A moist wound bed allows growth factors and various cell types, including epithelial cells, to migrate, facilitating wound edge contraction. Not surprisingly, grafts (e.g., wound dressings) play an important role in creating and maintaining such an environment. For example, grafts should be able to provide hydration to wounds that are dry or desiccated, to remove excessive fluid from the wound bed for wounds that excrete exudate, to facilitate oxygen diffusion into the wound, to conform to the wound bed and adhere to the skin, and to deliver pharmaceutical agents (e.g., antibiotics, antifungals, etc.,) at controlled and predictable rates over prolonged time periods.

The devices and methods disclosed herein are useful for producing grafts (e.g., wound dressings) configured to enhance healing of non-healing wounds. For example, in some embodiments, the devices described herein permit the graft (e.g., wound dressing) to be actively printed onto the wound bed in real time, thus ensuring that the graft conforms to the shape of the wound. Additionally, in some embodiments, the devices disclosed herein are configured to print one or more materials, for example, a biological material and/or a synthetic material. This allows the materials properties of the graft to be finely tuned prior to printing, for example, to enable fluid retention and/or fluid removal from the wound bed or to balance adhesion of the graft with easy and atraumatic removal. In some embodiments, the devices preserve a native structure of the biological and/or synthetic material being printed, for example, the secondary structure of adipose tissue. In this way, the printed graft preserves the native biological activity of the printed material and aids in wound closure. Other advantages of using the devices and methods of the present disclosure include the ability to print drug-loaded grafts (e.g., antibiotics, antifungals, etc.) with finely tuned drug release profiles.

Aspects of the present disclosure generally relate to a device for the deposition of a biological material and/or a synthetic material onto the surface of a subject (e.g., for the deposition of graft or wound dressing onto a wound of a subject).

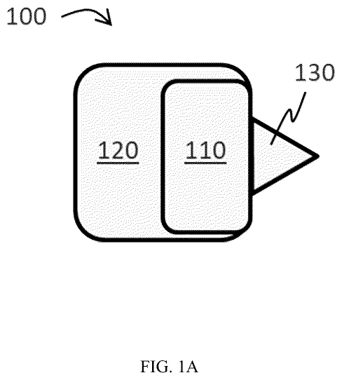

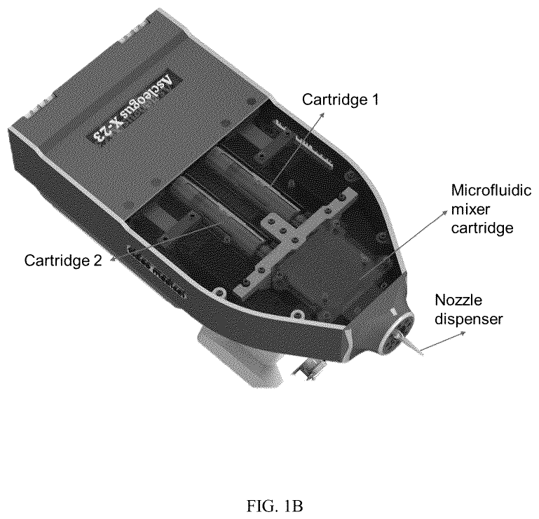

In some embodiments, the device comprises one or more reservoirs adapted to receive and/or contain a one or more biological materials or a one or more synthetic materials. For example, as illustrated in FIG. 1A, device 100 comprises reservoir 110. In some embodiments, device 100 comprises cooling element 120 adjacent the reservoir. In some embodiments, the cooling component is configured to keep an internal portion of the reservoir at particular temperature (e.g., greater than or equal to −30° C. and less than or equal to 10° C.), as described in more detail, below. In some embodiments, device 100 comprises dispenser 130 adjacent reservoir. In some embodiments, the dispenser is configured to dispense one or more biological materials and/or one or more synthetic materials onto the surface of the subject. In some embodiments, the biological materials and/or synthetic materials are in liquid form. In some embodiments, the biological materials and/or synthetic materials are in powder form. Biological materials and synthetic materials are described in more details, below.

A “subject” as used herein refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the self-righting article.

Some embodiments comprise printing a graft onto a wound of a subject. For example, in some embodiments, a method of printing a graft onto a wound of a subject comprises contacting a device to the wound and moving said device across said wound in a user-defined path; and depositing one or more biological materials or one or more synthetic materials, from the device onto the surface of said wound as the device moves along the user-defined path.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art.

In some embodiments, the device comprises one or more reservoirs. Any suitable number of reservoirs may be used in the devices contemplated herein. For example, in some embodiments, the device comprises greater than or equal to 1 reservoir, greater than or equal to 2 reservoirs, greater than or equal to 3 reservoirs, greater than or equal to 4 reservoirs, greater than or equal to 5 reservoirs, greater than or equal to 6 reservoirs, greater than or equal to 7 reservoirs, greater than or equal to 8 reservoirs, greater than or equal to 9 reservoirs, and greater than or equal to 10 reservoirs. In some embodiments, the device comprises less than or equal to 10 reservoirs, less than or equal to 9 reservoirs, less than or equal to 8 reservoirs, less than or equal to seven reservoirs, less than or equal to 6 reservoirs, less than or equal to 5 reservoirs, less than or equal to 4 reservoirs, less than or equal to 3 reservoirs, and less than or equal to 2 reservoirs. Combinations of the above-referenced ranges are also possible (e.g., the device comprises greater than or equal to 1 reservoir and less than or equal to 10 reservoirs).

In some embodiments, the one or more reservoir is adapted to receive and/or contain a biological material or a synthetic material. In some embodiments, the biological material maybe a first biological material, a second biological material, a third biological material, a fourth biological material, a fifth biological material, a sixth biological material, a seventh biological material, an eighth biological material, a nineth biological material, or a tenth biological material. In some embodiments, the synthetic material maybe a first synthetic material, a second synthetic material, a third synthetic material, a fourth synthetic material, a fifth synthetic material, a sixth synthetic material, a seventh synthetic material, an eighth synthetic material, a nineth synthetic material, or a tenth synthetic material.

For example, in some embodiments, the one or more reservoir is adapted to receive and/or contain a first biological material or a first synthetic material. In some embodiments, the one or more reservoirs comprises a second reservoir adapted to receive and/or contain a second biological material or a second synthetic material. Other combinations are also possible according to other embodiments. For instance, in some embodiments, the first reservoir comprises a first biological material or a first synthetic material. In some embodiments, the first reservoir comprises a second biological material or a second synthetic material. In some embodiments, the second reservoir comprises a first biological material or a first synthetic material. In some embodiments, the second reservoir comprises a second biological material or a second synthetic material.

Those of ordinary skill in the art would be capable of selecting suitable materials for the biological material (e.g., the first and/or second biological material) based upon the teachings of this specification. For example, in some embodiments, the first and/or second biological material is selected from the group consisting of adipose tissue, placental tissue, placental components, platelet rich plasma (PRP), keratin, plasma components, human cadaver proteins, and animal xeno proteins. In some embodiments, the first and/or second biological material comprises platelets.

In some embodiments, the first and/or second biological material comprises adipose tissue, or any fragment thereof, from an allogenic source (e.g., adipose tissue obtained from a person different than the subject). In some embodiments, the first and/or second biological material comprises adipose tissue, or any fragment thereof, from an autologous source (e.g., adipose tissue obtained from the subject)

In some embodiments, human cadaver proteins comprise a structural protein. In some embodiments, the structural protein is a mammalian structural protein (e.g., collagens type I, III, IV, V, VII, laminin-1, laminin-5, fibronectin, Keratin etc.). In some embodiments, the protein is a growth factor (e.g., EGF, EGF R, EG-VEGF, FGF-4, FGF-7, PDGF-AA, PIGF, VEGF, VEGF R2, VEGF R3, VEGF-D, ICAM-1, bFGF, KGF, TGF, HGF, NGF, IGFBP-3, IGFBP-4, IGFBP-6, AR, MIP-1a, MIP-1b, MIP-1d, SCF, SCF R, BDNF, BMP-4, BMP-5, b-ngf, GDNF, OPG, MIG, GH, Insulin, MCSF, MCSF R, TGFa, TGFb3, etc.). In some embodiments, the proteins is a cytokine (e.g., IR-IRA, IL-la, IL-1b, IL-Ira, IL-6sR, TNF, TNFb, TNFa, I-309, NT-3, NT-4, BLC, MCP-1, IL-12p40, IL-16, IL-17, IL-4, IL-6, IL-8, IL-10, TIMP-1, TIMP-2, TIMP-4, etc.)

In some embodiments, mammalian proteins comprise collagen type I, type IV, fibronectin, and keratin.

In some embodiments, a first and/or second synthetic material is a synthetic analog of anyone of the biological materials disclosed herein. For example, in some embodiments, the synthetic material comprises a synthetic analog of adipose tissue, placental tissue, placental components, platelet rich plasma (PRP), keratin, plasma components, human cadaver proteins, and animal xeno proteins. In some embodiments, the first and/or second synthetic material comprises platelets. The synthetic analog may comprise the entire biological material, a fragment of the biological material, or a combination of fragments of the biological material.

In some embodiments, the first and/or second biological material has been processed into a powder. In other embodiments, the first and/or second synthetic material has been processed into a powder. In some embodiments, the powder is dispersed within a solution to form a suspension. In some embodiments, the solution is capable of being extruded, for example, using any one of the devices disclosed herein.

In some embodiments, the first and/or second biological material or the first and/or second synthetic material is processed prior to deposition using the devices of the present disclosure. In some cases, the first and/or second biological material or the first and/or second synthetic material has been homogenized. Any suitable method of homogenizing said materials known to the skilled artisan may be used according to the methods disclosed herein. For example, in some cases the first and/or second biological material or the first and/or second synthetic material are homogenized using a high-speed homogenizer. Those of skill in the art will appreciate that the polydispersity index (PDI) of the homogenized material can be controlled, for example, by varying the type of homogenizer (e.g., piston homogenizer, rotor stator homogenizer, ultrasonic homogenizer, bead mill homogenizer, high-pressure homogenizer, paddle blenders, and mortar and pestle homogenizers) and the relevant parameters for each homogenizer. For example, for rotor-stator type homogenizers, the PDI can be altered by changing the rotor-stator design and the homogenizer speed.

In some embodiments, the first and/or second material or the first and/or second synthetic material has a PDI of between 1 and 20. In some embodiments, the PDI is greater than or equal to 1, greater than or equal to 2, greater than or equal to 3, greater than or equal to 4, greater than or equal 5, greater than or equal to 6, greater than or equal to 7, greater than or equal to 8, greater than or equal to 9, greater than or equal to 10, greater than or equal to 12, greater than or equal to 14, greater than or equal to 16, greater than or equal to 18, or greater than or equal to 20. In some embodiments, the PDI is less than or equal to 20, less than or equal to 18, less than or equal to 16, less than or equal to 14, less than or equal to 12, less than or equal to 10, less than or equal to 9, less than or equal to 8, less than or equal to 7, less than or equal to 6, less than or equal to 5, less than or equal to 4, less than or equal to 3, less than or equal to 2, or less than or equal to 1.

In some embodiments, the device comprises a cooling component. Those of ordinary skill in the art would be capable for selecting a cooling component for use with the devices described herein based upon the teachings of this specification. For example, in some embodiments, the cooling component comprises a Peltier water jacket. In some embodiments, the cooling component is selected from the group consisting of a cold plate, a heat sink, a pump, enclosure coolers, air-to liquid and liquid-to-liquid heat exchangers, and combinations thereof.

In some embodiments, the cooling component is adjacent the one or more reservoirs. In some embodiments, the cooling component is configured to keep an internal portion of the one or more reservoirs at greater than or equal to −30° C. and less than or equal to 10° C. For example, in some embodiments, the cooling component is configured to keep an internal portion of the one or more reservoirs at a temperature greater than or equal to −30° C., greater than or equal to −20° C., greater than or equal to −10° C., greater than or equal to −8° C., greater than or equal to −6° C., greater than or equal to −4° C., greater than or equal to −2° C., greater than or equal to 0° C., greater than or equal to 2° C., greater than or equal to 4° C., greater than or equal to 6° C., or greater than or equal to 8° C. In some embodiments, the cooling component is configured to keep an internal portion of the one or more reservoirs at a temperature less than or equal to 10° C., less than or equal to 8° C., less than or equal to 6° C., less than or equal to 4° C., less than or equal to 2° C., less than or equal to 0° C., less than or equal to −2° C., less than or equal to −4° C., less than or equal to −6° C., less than or equal to −8° C., or less than equal to −9° C., less than or equal to −10° C., or less than or equal to −20° C. Combinations of the above-referenced ranges are also possible (e.g., greater than or equal to −30° C. and less than or equal to 10° C.). Other ranges are also possible.

In some embodiments, the cooling component is configured to maintain the temperature of the internal portion (e.g., at a temperature greater than or equal to −10° C. and less than or equal to 10° C.) of the reservoir for greater than or equal to 2 minutes and less than or equal to 60 minutes. For example, in some embodiments, the cooling component maintains the temperature of the internal portion of the reservoir for greater than or equal to 2 minutes, greater than or equal to 4 minutes, greater than or equal to 6 minutes, greater than or equal to 8 minutes, greater than or equal to 10 minutes, greater than or equal to 20 minutes, greater than or equal to 30 minutes, greater than or equal to 40 minutes, greater than or equal to 50 minutes, or greater than or equal to 60 minutes. In other embodiments, the cooling component maintains a temperature of the internal portion of the reservoir for less than or equal to 60 minutes, less than or equal to 50 minutes, less than or equal to 40 minutes, less than or equal to 30 minutes, less than or equal to 20 minutes, less than or equal to 10 minutes, less than or equal to 8 minutes, less than or equal to 6 minutes, less than or equal to 4 minutes, or less than or equal to 2 minutes.

Other configurations are also possible according to other embodiments. For example, in some embodiments, the cooling component is positioned adjacent an exterior wall of the device. In some embodiments, the cooling component is positioned adjacent an interior wall of the device. In other embodiments, the cooling component is positioned adjacent the mixing component. Without wishing to be bound by any particular theory, it is believed that positioning the cooling component adjacent to the mixing component solidifies the first and/or second biological material as it is deposited from the device. Additionally, or alternatively, positioning the cooling component adjacent to the mixing component solidifies the first and/or second synthetic material as it is deposited from the device.

In some embodiments, the device further comprises a mixing component. Any suitable mixing component known to the skill artisan may be used to mix any of the contents of the one or more reservoirs prior to printing the graft. For example, in some embodiments, the mixing component comprises an active mixing component (e.g., uses mechanical mixing). In other embodiments, the mixing component comprises a passive mixing component (e.g., a microfluidic mixer).

In some embodiments, the mixing component comprises a microfluidic mixer. In some embodiments, the microfluidic mixer comprises one or more microfluidic channels with one or more defined geometries. For instance, in some embodiments, the one or more defined geometries includes a sequential convergence-divergence geometry. In other embodiments, the one or more defined geometries includes a serpentine geometry. Alternatively, or additionally, in some embodiments, the one or more defined geometries includes a combination of a sequential convergence-divergence geometry and a serpentine geometry. Other geometries are also possible according to other embodiments. For example, in some embodiments, the one or more defined geometries includes a rastering-type geometry. In other embodiments still, the one or more defined geometries comprise cross-sectional area of any continuous space filling geometry, including, but not limited to circle, triangle, rectangle, rhombus, square, and trapezoid.

In some embodiments, the mixing components configured to mix the contents of two or more reservoirs. For example, in some embodiments, the mixing component is configured to mix a first volume of the first reservoir with a second volume of the second reservoir. However, it should be understood that the mixing component contemplated herein has the ability to mix more than two reservoirs. In some embodiments, the mixing component is configured to mix the contents of greater than or equal to 2 reservoirs, greater than or equal to 3 reservoirs, greater than or equal to 4 reservoirs, greater than or equal to 5 reservoirs, greater than or equal to 6 reservoirs, greater than or equal to 7 reservoirs, greater than or equal to 8 reservoirs, greater than or equal to 9 reservoirs, and greater than or equal to 10 reservoirs. In other embodiments, the mixing component is configured to mix the contents of less than or equal to 10 reservoirs, less than or equal to 9 reservoirs, less than or equal to 8 reservoirs, less than or equal to 7 reservoirs, less than or equal to 6 reservoirs, less than or equal to 5 reservoirs, less than or equal to 4 reservoirs, or less than or equal to 3 reservoirs.

In some embodiments, the mixing component mixes the first volume of the first material to the second volume of the second material at a ratio of between 1% to 99% (v/v). In some embodiments, the mixing component mixes the first volume of the first reservoir to the second volume of the second reservoir at a ratio of between 10% and 90% (v/v), between 20% and 80% (v/v), between 30% and 70% (v/v), between 40% and 60% (v/v), between 50% and 50% (v/v), between 60% and 40% (v/v), between 70% and 30% (v/v), between 80% and 20% (v/v), or between 90% and 10% (v/v), respectively.

However, in some embodiments, the mixing component, as contemplated herein, is configured to mix the contents of more than two reservoirs in any ratio. Those of skill in the art will appreciate that the sum of the ratios must equal 100% on volume or mass basis. For example, in some embodiments, the mixing component is configured to mix the contents of a first reservoir, a second reservoir, and a third reservoir at a ratio of 25%, 25%, and 50% (v/v), respectively.

In some embodiments, the devices of the present disclosure further comprise a transfer mechanism to permit aseptic loading of the first and/or second biological material or the first and/or second synthetic material into the one or more reservoirs. Any suitable transfer mechanism known to the skill artisan may be used in any of the devices disclosed herein. For example, in some embodiments, the devices comprise a two-latch system. In some embodiments, a first latch of the two-latch system, is opened and two syringe reservoirs (e.g., filled with the desired biological material) are added to the device. In some embodiments, a second latch, of the two latch system, is used to connect the syringe reservoirs to the microfluidic mixer and the dispenser.

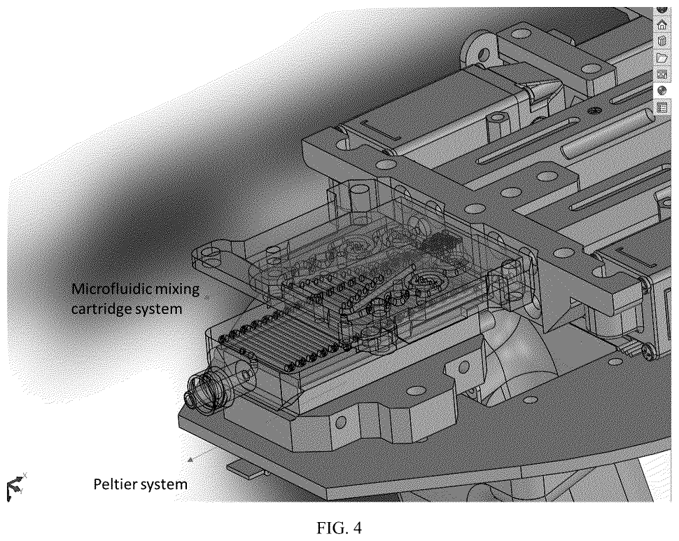

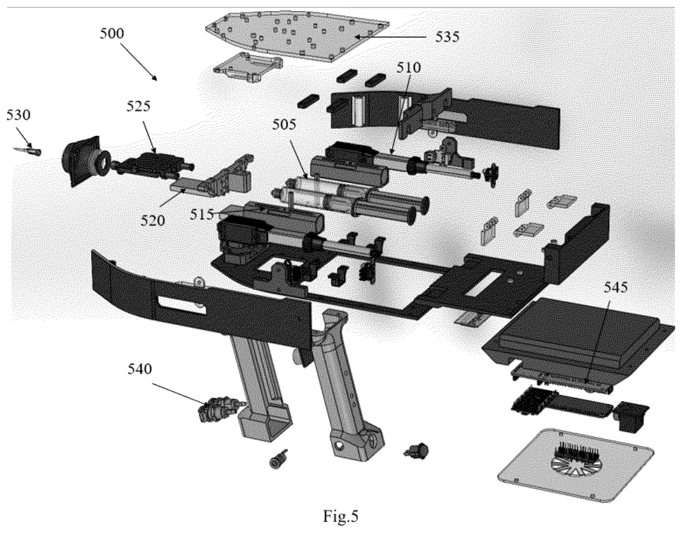

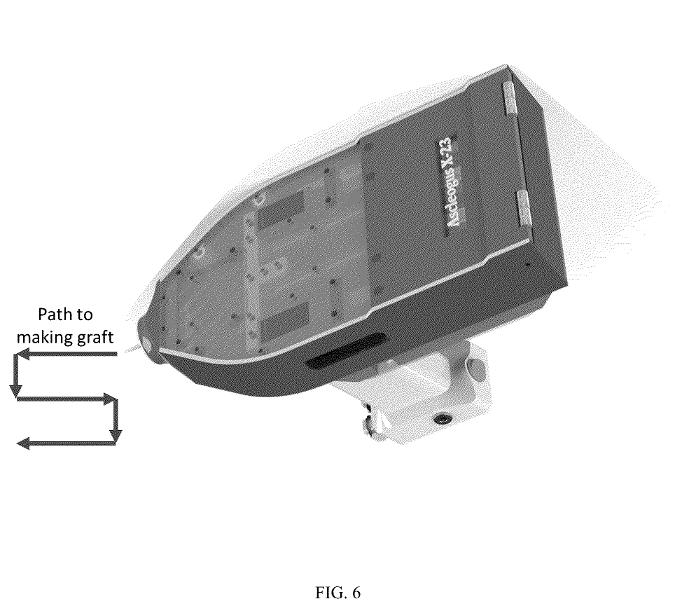

In some embodiments, the devices of the present disclosure further comprise a dispenser, for example, to deposit the contents of the one or more reservoirs onto a surface (e.g., a wound bed). In some embodiments, the dispenser is attached to the mixing cartridge (see FIGS. 5 and 6). In some embodiments, the dispenser is positioned adjacent the reservoir (see FIG. 7). In some embodiments, the dispenser is configured to dispense the biological material or the synthetic material onto a surface. In some embodiments, the surface is the surface of the subject (e.g., a wound).

In some embodiments, the device further comprises a nozzle. Any suitable nozzle geometry known to the skilled artisan may be used in any of the devices disclosed herein. In some embodiments, the nozzle geometry is selected from the group consisting of a concentric geometry, side-by-side geometry, star pattern encircle geometry, and serpentine encircle geometry. In some embodiments, the nozzle may have any suitable cross-sectional shape including circular, square, triangle, diamond, etc. In some embodiments, the nozzle may comprise an outer layer having a first shape and an inner layer having a second shape selected from the cross-sectional shapes described above. For example, in some embodiments, the nozzle has a first layer having a star shaped cross-section and a second layer having a circular cross-sectional shape. In some embodiments, more than one nozzle may be present, and each nozzle may have a suitable cross-sectional shape.

In some embodiment, the nozzle geometry is selected from a circular, conical luer lock with various diameter ranging from between 100 microns to 2 mm. In some embodiments, the nozzle diameter is greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1 mm, or greater than or equal to 2 mm. In some embodiments, the nozzle diameter is less than or equal to 2 mm, less than or equal to 1 mm, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, or less than or equal to 100 microns.

In some embodiments, the device further comprises a power source. In some embodiments, the power source is selected from the group consisting of an external electrical source, electromechanical system, solar power, a battery, or combination thereof.

In some embodiments, the device further comprises a drive train arrangement. Those of skill in the art will appreciate that the drive train arrangement may an electric drive train arrangement or a non-electric drive train assembly. Thus, in some embodiments, the device comprises an electric drive train arrangement. In some embodiments, the electric drive train arrangement is powered with direct-current (DC). In other embodiments, the electric drive train is powered with alternating current (AC). In other embodiments still, the drive train arrangement is powered using an alternative energy source such as manual power or solar power. In some embodiments, the drive train arrangement is used to control one or more functions of the device, such as for example, deposition rate, and/or the mixing ratio.

FIG. 5 shows an exemplary device 500 comprising disposable syringes 505 placed into copper gasket 515. Dispenser 530, of the syringe, is connected to a drive train arrangement connected to a stepper motor with screw system 510, which is connected to Peltier system 520. The Peltier system is attached to the bottom of microfluidic mixing cartridge 525 which is then connected to dispenser 530 to extrude biological or synthetic material on to a surface. In some embodiments, a latch is used to open the area of the syringe system that is protected by plastic cover 535. In some embodiments, the entire system is electromechanically controlled using electronics system 545. In some embodiments, a cooling liquid is used to cool down the temperature of Peltier system 520 using cooling inlets 540.

FIG. 7B shows an exemplary device 700 comprising dispenser 705 connected to drive train arrangement 710. The drive train arrangement comprises first end 715 that locks onto the end of the dispenser 705, a micro-stepper motor 720, and support arm 725 that connects the first end 715 to micro-stepper motor 720. In this way, the drive train assembly controls retraction of the dispenser, for example, for aseptic loading of a biological or synthetic material into one or more reservoirs, and compression of the dispenser, for example, for depositing the biological or synthetic material onto a surface. In some embodiments, the device comprises Peltier system 730 configured to solidify the graft via freezing.

In some embodiments, the device further comprises an electronic control circuit. Without wishing to be bound by any particular theory, an electronic control circuit is known in the art as a circuit that carries the electric signals directing performance of a control device but does not carry the power that the device controls. Thus, in some embodiments, the electronic control circuit controls one or more functions of the device, such as for example, the device temperature, deposition rate, and/or the mixing ratio. In some embodiments, the electronic control circuit controls the drive train assembly, as described elsewhere herein. In some embodiments, the electronic control circuit comprises a microelectromechanical system (MEMS). In some embodiments, the MEMS is configured to control one or more device functions selected from the group consisting of device temperature, deposition rate, and mixing ratio.

In some embodiments, devices disclosed herein comprise a graphical user interface (GUI). Any GUI software known to the skill artisan may be used in any of the devices disclosed herein. In some embodiments, the GUI is configured to permit a user to control one or more functions of the device. In some embodiments, the GUI is configured to interface with one or more electronic control circuits. In some embodiments, the GUI is configured to interface with the MEMS. For example, in some embodiments, the GUI is configured to permit a user to control the deposition of the first and/or second biological material. In other embodiments, the GUI is configured to permit a user to control the deposition of the first and/or second synthetic material. In other embodiments, the GUI is configured to permit a user to control the device temperature. In some embodiments, the GUI is configured to permit a user to control the device mixing ratio.

Other aspects of the present disclosure relate to methods of printing a graft using any one of the devices disclosed herein. In some embodiments, the methods comprise printing a graft onto a wound of the subject. The wound may be any wound that needs to be treated with a graft, for example, a nonhealing wound. Nonlimiting examples of wounds that may be treated using the devices disclosed herein include diabetic ulcers, pressure ulcers, venous ulcers, arterial insufficiency ulcers, surgical wounds, burns, and abrasions. Other wounds are also possible in other embodiments.

In some embodiments, the methods comprise contacting any one of the devices disclosed herein onto a wound of a subject and moving the device across the wound in a user-defined path. Any suitable user-defined path can be used in the methods disclosed herein. For example, in some embodiments, the user-defined path is selected from the group consisting of concentric circles, concentric squares, concentric triangles, concentric ovals, a spiral path, a zigzag path, a serpentine path, or the like. In some embodiments for example the user-defined path is any continuous space filling path.

In some embodiments, the methods comprise depositing a biological material or a synthetic material from the device onto the surface of the wound as the device moves along the user-defined path. In some embodiments, the methods comprise depositing one or more biological materials and/or one or more synthetic materials from the device onto the surface of the wound as the device moves along the user-defined pathway. For example, in some embodiments, the methods comprise depositing a first biological material and/or a first synthetic material from the device onto the surface of the wound as the device moves along the user-defined path. In other embodiments, the methods comprise depositing a first biological material and a second biological material onto the surface of the wound as the device moves along the user-defined path. Additionally, or alternatively, the methods may comprise depositing a first synthetic material and a second synthetic material onto the surface of the wound as the device moves along the user-defined path. The above discussion is not intended to be limiting in any way as to the number of materials that can be deposited or the combination with which they are deposited onto the surface. The skilled artisan will appreciate that any suitable number and combination of biological and/or synthetic materials may be printed using the methods disclosed herein.

In some embodiments, the methods comprise using the device to maintain the one or more biological materials and/or one or more synthetic materials at a temperature of greater than or equal to −30° C. and less than or equal to 10° C. prior to deposition. Without wishing to be bound by any particular theory, it is generally believed that maintaining the one or more biological materials or the one or more synthetic materials at a temperature of greater than or equal to −30° C. and less than or equal to 10° C. prior to deposition preserves the native tissue structure of the biological material and/or the synthetic material after deposition.

In some embodiments, the methods comprise increasing the temperature of the one more biological material and/or the one or more synthetic materials to greater than or equal to 10° C. following deposition from the device onto the surface of the wound. Any suitable method may be used to increase the temperature of the one or more biological materials and/or one or more synthetic materials to greater than or equal to 10° C. following deposition from the device onto the surface of the wound. In some embodiments, thermal energy from the subject is used to increase the temperature of the graft to greater than or equal to 10° C. following deposition from the device onto the surface of the wound.

In some embodiments, the methods comprise mixing one or more biologically active agents with the one or more biological materials and/or the one or more synthetic materials contained within the one or more reservoirs of the device prior to printing the graft. In some embodiments, the biologically active agent is mixed with a first and/or second biological material prior to depositing the first and/or second biological material onto the wound. In other embodiments, the biologically active agent is mixed with the first and/or second synthetic material prior to depositing the first and/or second synthetic material onto the wound. In some embodiments, the biologically active agent is selected from the group consisting of an antibiotic, an antifungal agent, an anti-inflammatory, and pH buffering agent. Any suitable antibiotic, antifungal, anti-inflammatory, and pH buffering agent known to the skill artisan may be used in the methods disclosed herein.

In some embodiments, printing the one or more biological materials and/or the one or more synthetic materials comprising the one or more biologically active agents embeds the one or more biologically active agents within the printed graft. In some embodiments, the one or more embedded biologically active agents may release from the graft at controlled rates over a controlled time period.

In some embodiments, the methods of printing a graft onto a wound of a subject as described herein produces a hydrated graft. In some embodiments, the hydrated graft has a Young's modulus of between 5 kPa and 5 MPa. In some embodiments, the Young's modulus is greater than or equal to 5 kPa, greater than or equal to 10 kPa, greater than or equal to 20 kPa, greater than or equal to 40 kPa, greater than or equal to 80 kPa, greater than or equal to 160 kPa, greater than or equal to 320 kPa, greater than or equal to 640 kPa, greater than or equal to 1.2 MPa, greater than or equal to 2.5 MPa, or greater than or equal to 5 MPa. In other embodiments, the Young's modulus is less than or equal to 5 MPa, less than or equal to 2.5 MPa, less than or equal to 1.2 MPa, less than or equal to 640 kPa, less than or equal to 320 kPa, less than or equal to 160 kPa, less than or equal to 80 kPa, less than or equal to 40 kPa, less than or equal to 20 kPa, less than or equal to 10 kPa, and less than or equal to 5 kPa.

### EXAMPLES

The following examples are intended to illustrate certain embodiments described herein, including certain aspects of the present invention, but do not exemplify the full scope of the invention.

Example 1. FIG. 1 generally relates to a device comprising two reservoirs, a mixing component, and the dispensing nozzle. In some embodiments, FIG. 1 illustrates the interface between a first reservoir and second reservoir with the mixing component. In some embodiments, FIG. 1B further illustrates the interface between the mixing component and the dispenser.

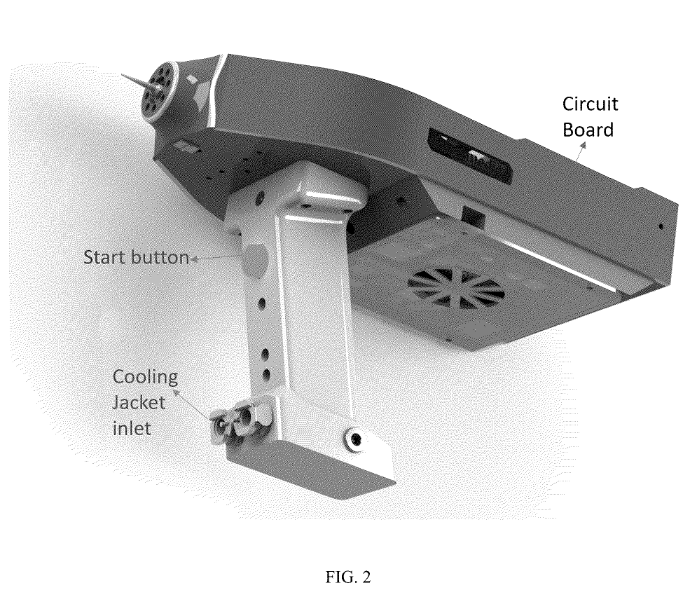

Example 2. FIG. 2 generally relates to a fully assembled device, as contemplated herein. In some embodiments, the fully assembled device comprises one or more reservoirs, a mixing component, a cooling component, a dispenser, control circuitry, and a motor.

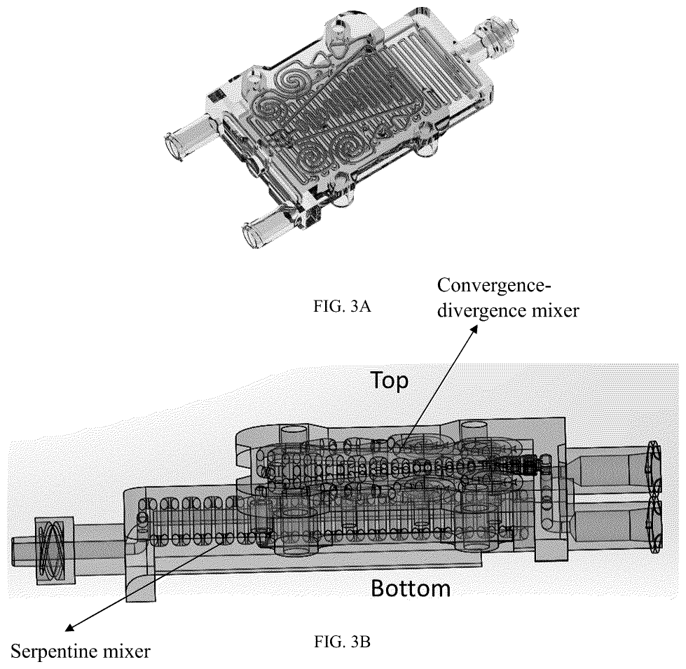

Example 3. FIG. 3 generally relates to exemplary mixing components as contemplated herein. In some embodiments, FIG. 3A illustrates a sequential convergence-divergence mixer. In some embodiments, FIG. 3B further illustrates a serpentine mixer.

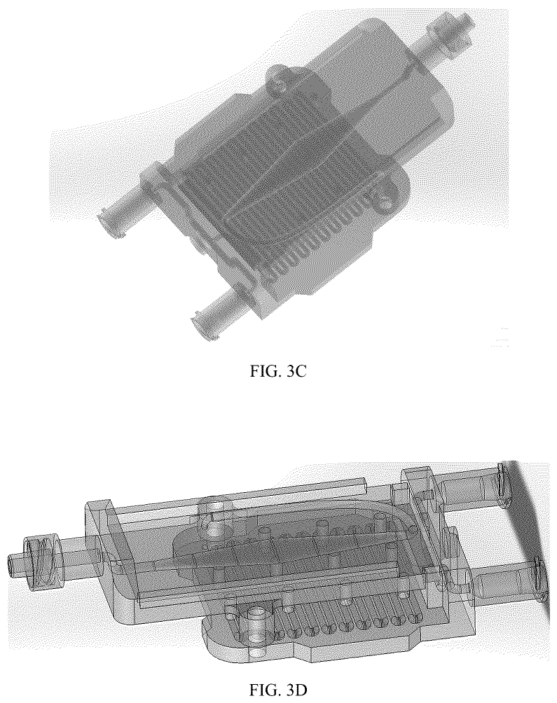

Example 4. FIG. 4 generally relates to an exemplary device comprising the mixing component adjacent the cooling system. In some embodiments, the cooling component is configured to keep an internal portion of the reservoir at greater than or equal to −30° C. and less than or equal to 10° C. In some embodiments, depositing a biological material or a synthetic material under these conditions preserves the native tissue structure of the biological material after deposition.

Example 5. FIGS. 5-6 illustrates an exploded view of a to an exemplary handheld device comprising two reservoirs, according to some embodiments.

Example 6. FIGS. 7A-7B generally relate to an exemplary device comprising a single reservoir, according to some embodiments. For example, FIG. 7A and 7B show top and cross-sectional views, respectively, of an exemplary device comprising a single reservoir.

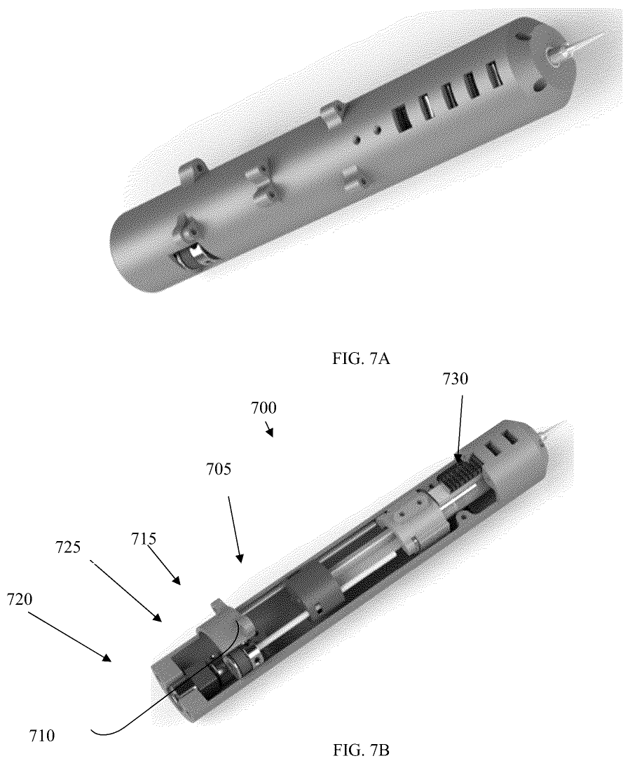

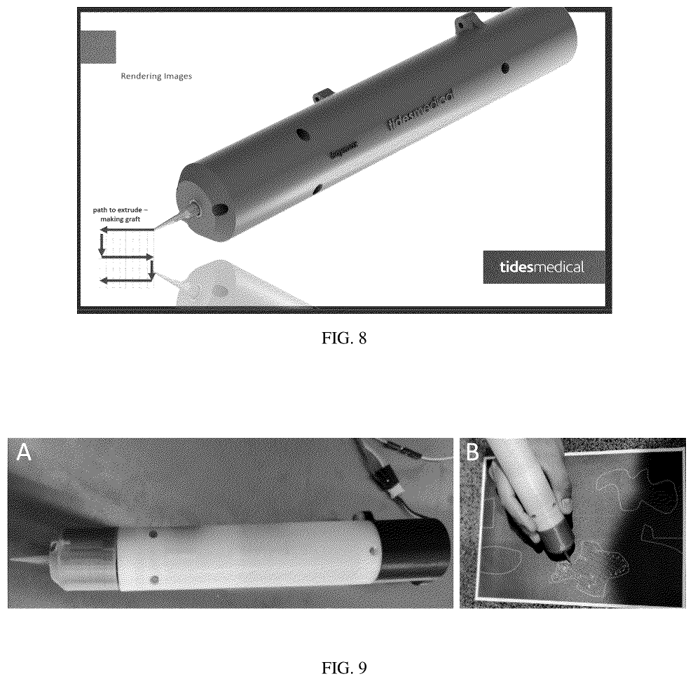

Example 7. FIGS. 8 and 9 generally relate to printing a graft using the devices contemplated herein. FIG. 8 illustrates the use of an exemplary device to print a graft using a user defined path. In the instant example, the path is a serpentine path. FIG. 9 shows a functioning prototype of an exemplary device contemplated herein. FIG. 9 also shows the device being used to print an adipose graft at about 4° C., according to some embodiments.

### Equivalents and Scope

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, gomboc, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, cast, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device for the deposition of a biological material and/or synthetic material onto a surface of a subject, the device comprising:
one or more reservoirs adapted to receive and/or contain a one or more biological materials or a one or more synthetic materials;
a cooling component adjacent the one or more reservoirs, wherein the cooling component is configured to keep an internal portion of the one or more reservoirs at greater than or equal to −30° C. and less than or equal to 10° C.; and
a dispenser adjacent the one or more reservoirs, wherein the dispenser is configured to dispense the one or more biological materials or the one or more synthetic materials onto the surface of the subject.

2. A method of printing a graft onto a wound of a subject, the method comprising:
contacting a device to the wound and moving said device across said wound in a user-defined path; and
depositing one or more biological materials or one or more synthetic materials, from the device onto the surface of said wound as the device moves along the user-defined path,
wherein the device is configured to maintain the one or more biological materials or the one or more synthetic materials at a temperature of greater than or equal to −30° C. and less than or equal to 10° C. prior to deposition.

3. The device as in claim 1, wherein the one or more reservoirs comprises a first and/or second reservoir adapted to receive and/or contain a first and/or second biological material or a first and/or second synthetic material.

4. The device as in claim 1, wherein the device further comprises a mixing component configured to mix a first volume of a first reservoir with a second volume of the second reservoir.

5. The device as in claim 1, wherein the mixing component comprises one or more microfluidic channels with one or more defined geometries.

6. The device as in claim 1, wherein the one or more defined geometries includes a sequential convergence-divergence geometry.

7. The device as in claim 1, wherein the one or more defined geometries includes a serpentine geometry.

8. The device as in claim 1, wherein the one or more defined geometries includes a combination of a sequential convergence-divergence geometry and a serpentine geometry.

9. The device as in claim 1, wherein the one or more defined geometries includes a rastering geometry.

10. The device as in claim 1, wherein the geometry comprises any continuous space filing geometry.

11. The device as in claim 1, wherein the mixing component mixes the first volume of the first reservoir and the second volume of the second reservoir at a ratio of between 1% to 99% (v/v).

12. The device as in claim 1, wherein the mixing component mixes the first volume of the first reservoir and the second volume of the second reservoir at a ratio of between 10% and 90% (v/v).

13. The device as in claim 1, wherein the mixing component mixes the first volume of the first reservoir and the second volume of the second reservoir at a ratio of between 40% and 60% (v/v).

14. The device as in claim 1, wherein the first reservoir comprises a first biological material or a first synthetic material.

15. The device as in claim 1, wherein the first reservoir comprises a second biological material or a second synthetic material.

16. The device as in claim 1, wherein the second reservoir comprises a first biological material or a first synthetic material.

17. The device as in claim 1, wherein the second reservoir comprises a second biological material or a second synthetic material.

18. The device as in claim 1, wherein the first and/or second biological material is selected from the group consisting of adipose tissue, placenta, placenta components, platelet-rich plasma (PRP), plasma components, keratin, human cadaver proteins, animal xeno proteins, and bovine protein.

19. The device as in claim 1, wherein the first and/or second biological material comprises adipose tissue, or any fragment thereof, from autologous source.

20. The device as in claim 1, wherein the first and/or second material comprises adipose tissue, or any fragment thereof, from allogeneic source.

21-55. (canceled)
